(19) United States
(12) Patent Application Publication
(10) Pub. No.: US 2010/0179588 A1
(43) Pub. Date: Jul. 15, 2010

(54) VASCULAR PUNCTURE CLOSURE SYSTEM WITH GUIDE SHEATH STABILIZER

(75) Inventors: Ghaleb Sater, Acton, MA (US); Thierry Benjamin, Lowell, MA (US); Timothy J. Wood, Waltham, MA (US); Richard Lobello, Johnston, RI (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/353,624

(22) Filed: Jan. 14, 2009

(51) Int. Cl. A61B 17/10

(52) U.S. Cl. 606/213

(57) ABSTRACT

A guide sheath for use in guiding a vascular puncture closure device to and in registry with a vascular puncture includes a stabilization system having retention wires deployable from the sheath and engageable with the vessel wall. The retention wires pierce the vessel wall at spaced locations, proximally and distally, about the puncture and form anchors to retain the sheath in registry with the puncture.

### FIELD OF THE INVENTION

The invention relates to systems and techniques for closing a percutaneous puncture in a blood vessel at the conclusion of an intravascular procedure.

### BACKGROUND

Various cardiovascular procedures, such as angioplasty and stent placement, among others, are performed by inserting into and manipulating within a patient's vasculature, wires and catheters adapted to perform those procedures. In coronary and other such intravascular interventional procedures access to the vasculature typically is percutaneous, often through the femoral artery, involving insertion of a needle in the region of the groin to form a track through subcutaneous tissue and to puncture and create an arteriotomy in the artery. A guidewire then is advanced through the needle and into the femoral artery. The needle then is removed and a dilator carrying an introducer sheath then is advanced over the guidewire, along the needle track and into the femoral artery. The dilator enlarges the track through the tissue and widens a puncture in the vessel so that it may receive subsequent catheters and the like. With the introducer sheath having been advanced into the vessel, the dilator is removed leaving the introducer sheath in place. The guidewire and introducer sheath serve as guides to provide access into the femoral artery, through the arteriotomy, for catheters or other instrumentalities in order to perform the selected procedure within the patient's vasculature.

After the intravascular procedure has been completed, the procedural devices are removed and the arteriotomy must be closed. A number of techniques are known to facilitate closure and healing of the arteriotomy. These include application of pressure at the puncture site, often for a relatively extended length of time until hemostasis is achieved, or the use of biological adhesives or plugs adapted to seal the arteriotomy, or the use of staples or clips. Some closure systems include an arrangement to engage the artery to temporarily draw the edges of the arteriotomy together while a final closure device, such as a stapler, sutures, adhesives or other means is used to effect the permanent closure of the arteriotomy. Some closure systems include a tubular guiding sheath that is percutaneously positioned through the enlarged needle track with a distal outlet opening of the guiding sheath disposed immediately adjacent the arteriotomy. With the sheath so positioned, a closure device can be advanced through the sheath to apply its closure element or procedure to the region of the arteriotomy to close it. After the device has performed its function and hemostasis has been achieved, the sheath and other elements of the closure system are removed.

In order for such a sheath-based system to be effective, it is important that the distal end of the sheath be stabilized in a fixed position relative to the vascular puncture. Proper positioning of the distal end of the sheath assures that the closure elements that are delivered through the sheath are directed to and are positioned properly with respect to the puncture and the vessel wall to be able to close the puncture. U.S. Pat. No. 6,755,842 to Kanner describes linear, wire-like members that are mounted to the sheath and may be advanced through the arteriotomy at laterally opposed edges or ends of the puncture. The distal portions of the wire-like elements are capable of reforming within the lumen of the blood vessel into an enlarged shape that functions to engage the inner luminal surface of the vessel so that they cannot be withdrawal through the puncture, thus serving to anchor the wires. The anchor elements thus retain the open distal end of the sheath in position about the arteriotomy to guide the vascular closure device into position. After the puncture wound has been closed or hemostasis otherwise achieved, the anchors are reverted to their linear configuration and are withdrawn together with the sheath and the closure device.

In some instances, it may be desirable to stabilize the position of the distal end of the sheath by means other than extending a wire or an anchor through the arteriotomy. It is among the general objects of the invention to provide such a system for stabilizing the guide sheath.

### SUMMARY OF THE INVENTION

The invention employs a guide sheath having a central lumen and anchors which are pre-formed on the distal ends of elongate wires carried in wire lumens of the sheath. The wires can be contained in a straight configuration within their lumens and can be extended from the distal end of the sheath to assume a pre-formed anchor shape to anchor to the vessel wall. The distal tips of the wires are sharp and are arranged to engage and pierce the wall of the blood vessel from outside the vessel at spaced locations relative to the puncture, e.g., proximal (downstream) and distal (upstream) of the vascular puncture, preferably along the midline of the blood vessel. The anchors retain and stabilize the sheath in position during the closure procedure in which a vessel puncture closure device may be guided to the puncture site through the central lumen to close the puncture while the anchors stabilize the position of the sheath. The anchors are retractable to a straight configuration into the sheath after the procedure has been completed. The sheath may have a control handle attached to its proximal end to control the functions of the device.

A blood marking tube is provided in the central lumen during the initial introduction and positioning of the device. The blood marking tube extends beyond the distal end of the sheath and includes a lumen by which the sheath and blood marking tube can be advanced over an indwelling guidewire through the needle track to guide the assembly to the region of the arteriotomy. The blood marking tube may have reduced diameter proximal and distal segments dimensioned to receive the guidewire in a close sliding fit and an intermediate region with a lumen large enough to permit blood flow when the guidewire is in place. A distal blood marking port is formed proximally of the distal segment and a proximal blood marking port is located distally of the reduced diameter proximal segment. The blood marking tube and sheath are cooperatively engaged at their proximal regions so that the distance between the distal blood marking port and the distal end of the sheath is fixed to enable the clinician to determine when the distal end of the sheath is positioned correctly. This may be accomplished by providing a limit stop associated with the control handle to limit the extent of distal movement of the blood marking tube relative to the sheath. The distal end of the sheath may be formed at an oblique angle to generally parallel the longitudinal axis of the vessel when the procedure involves percutaneous entry at an oblique approach angle.

The retention anchors are pre-formed on the ends of wires to have a selected non-linear shape when relaxed and are sufficiently flexible to assume a straight configuration when retracted into the wire lumens. The wires have sharp distal tips sufficient to pierce the vessel wall and extend into or through the vessel wall as they reform toward their non-linear anchoring shapes. In a preferred embodiment, the wire lumens may be located in the sheath to lie generally along a plane (referred to as a central plane) that parallels the vessel while intersecting tile arteriotomy when the distal face of the sheath is aligned and in registry with the outer surface of the vessel. The axes of the central lumen and wire lumens may be considered as lying along the central plane. Thus the wires, as they emerge from their lumens, can pierce the vessel wall along longitudinally spaced points proximally and distally of the arteriotomy with the distal ends forming the anchors within the vessel lumen. With the distal ends of the wires formed in their retentive anchoring shape to secure and stabilize the sheath about the region of the puncture, blood flowing out of the proximal end of the blood marking tube should be observed and maintained. The clinician can confirm that blood flow is maintained after applying a slight tug to the device before the blood marking tube and guide wire are withdrawn from the central lumen of the sheath. With the lumen of the sheath unobstructed, a closure device then may be advanced through the sheath, operated to perform its arteriotomy closing function and the retention wires may be withdrawn into their respective lumens. The sheath and closure device then may be removed from the patient.

### DESCRIPTION OF THE DRAWINGS

In the accompanying drawings in which components are not intended to be in scale and in some cases are in exaggerated scale for ease of explanation and illustration:

FIG. 1 is a diagrammatic illustration of an artery with a vascular puncture;

FIG. 2 is a diagrammatic illustration, in section, of the artery of FIG. 1 as seen along the line 2-2 of FIG. 1;

FIG. 3 is a diagrammatic illustration, in section, of the artery as seen along the line 3-3 of FIG. 1;

FIG. 4 is a diagrammatic illustration of an artery with a guidewire extending through the needle track (in phantom), the arteriotomy and into the lumen of the artery, after the intravascular procedure has been completed but before the arteriotomy has been closed;

FIG. 5 is a general illustration of a device as may be used in the practice of the invention;

FIG. 6 is a diagrammatic illustration of the distal end of a device such as shown in FIG. 5 in which the distal end of the sheath of the device has been positioned adjacent the arteriotomy but in which its position relative to the arteriotomy has not yet been stabilized;

FIG. 7 is an illustration similar to FIG. 6 in which hook-shaped anchors associated with the sheath have been engaged with the artery to stabilize the position of the sheath with respect to the arteriotomy;

FIG. 8 is an illustration similar to FIG. 7 but with the blood marking tube and guide wire having been removed from the sheath, leaving the sheath central lumen open in readiness to guide a closure device to the arteriotomy, with the closure device shown diagrammatically in phantom;

FIG. 8A is a diagrammatic end view of the distal face of a sheath as seen along the line 8A-8A of FIG. 8;

FIG. 9 is a fragmented longitudinal sectional illustration of a dilator that may be used in practicing the invention; and

FIG. 10 is a diagrammatic transverse sectional illustration of the sheath of the device as seen along the line 10-10 of FIG. 5;

FIG. 10A is an illustration similar to FIG. 10 of an alternate construction for the sheath;

FIG. 11 is at diagrammatic illustration of a sheath anchored to a blood vessel with the distal ends of the wires forming an alternative form of anchor;

FIG. 12 is a side view of the anchoring element of FIG. 11;

FIG. 13 is a front view of the alternative anchoring element as seen along the line 13-13 of FIG. 12.

FIG. 14 is a more detailed illustration of the control handle similar to that of FIG. 5;

FIG. 15 is an illustration of the control handle with the blood marking tube removed from and separated from the handle;

FIG. 16 is an exploded illustration of the components of the control handle;

FIG. 17A is a sectional illustration of the handle region as seen along a horizontal section plane and illustrating the components of the handle before the device is placed in use;

FIG. 17B is a sectional illustration of the handle as seen along the line 17B-17B of FIG. 17A with a guidewire extending through the device but before the device has been actuated;

FIG. 17C is an illustration similar to FIG. 17A but with the retractor assembly having been withdrawn proximally to release the spring-biased mechanism for driving the retention wires distally into engagement with the blood vessel;

FIG. 17D is a sectional illustration of the handle portion of the device as seen along the line 17D-17D of FIG. 17C;

FIG. 17E is a sectional illustration similar to FIG. 17C showing the release of the latch arrangement by which the proximal cap and blood marking tube can be released and retracted;

FIG. 17F is al illustration similar to FIG. 17E but with the wire block an(wires being re-cocked to enable the wires to be redeployed should the clinician have determined that the initial deployment was unsatisfactory;

FIG. 17G is an illustration similar to FIG. 17E in which the clinician has determined that the retention wires have properly engaged the arterial wall and with the proximal cap being separated from the handle;

FIG. 17H is a sectional illustration along a vertical plane of the device illustrating the manner in which a portion of the arteriotomy closure device engages with the retraction assembly to trigger retraction of the retention wires;

FIG. 17I is an illustration similar to FIG. 17H illustrating the manner in which operation of the arteriotomy closure device releases a latch on the handle to enable a compression spring to cause retraction of the retention wires; and

FIG. 17J is an illustration similar to FIG. 17I showing the configuration of the control handle when the retraction wires have been fully retracted.

### DESCRIPTION OF THE ILLUSTRATIVE EMBODIMENTS

In the description of the invention, “proximal” and “rearward” will refer to a direction away from the patient, that is, toward the operator of the device, and “distal” and “forward” will refer to the opposite direction, away from the clinician.

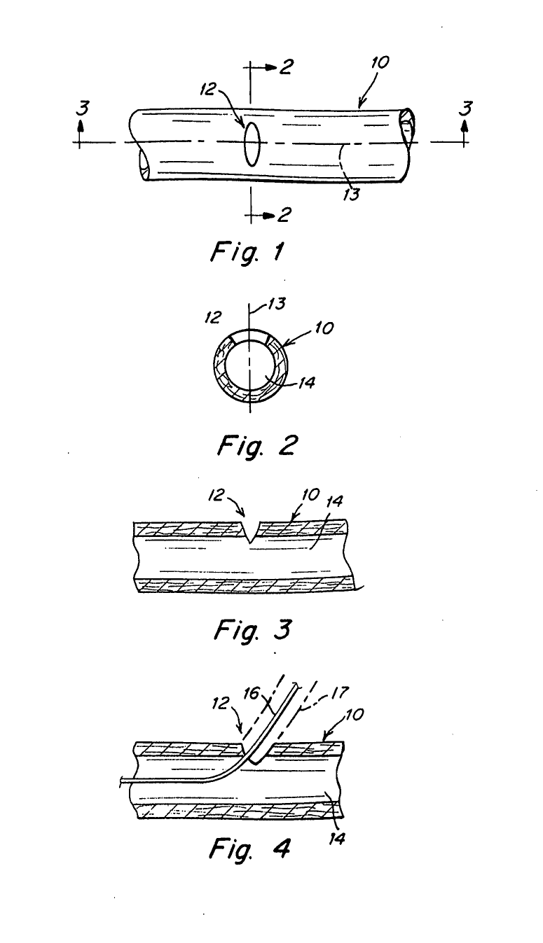

FIGS. 1-4 illustrate, diagrammatically, a segment of an artery 10 that has been punctured by a needle to form an arteriotomy 12 through which various wires, catheters and the like may be advanced and guided into the lumen 14 of the vessel in order to perform any of a variety of well-known intravascular procedures. Skin and layers of tissue surrounding artery 10 are also punctured by the needle to form a track leading to arteriotomy 12. However, these additional tissues are omitted to enhance clarity of the figures. FIGS. 1 and 2 illustrate diagrammatically a typical configuration of an arteriotomy in which the puncture takes the form of a slit that extends in a circumferential or transverse direction, generally perpendicular to the length of the vessel. The circumferential orientation results from the muscle structure of the artery in which the muscle fibers extend generally circumferentially. Typically, the needle puncture that initiates the arteriotomy is followed by subsequent, larger diameter instruments that progressively dilate the dimensions of the arteriotomy 12 to be able to accept the larger intravascular devices. For ease of explanation, the punctured vessel may be considered as having a midline plane 13 (FIGS. 1 and 2) that extends along the length of the vessel. The transverse slit that forms the arteriotomy is substantially perpendicular to midline plane 13. Ideally, the arteriotomy is centered laterally in the artery wall such that the midportion of the slit intersects the midline plane 13.

Typically, when initiating a percutaneous procedure, the needle is advanced at an oblique approach angle to the blood vessel and consequently, the needle track and subsequently inserted wire catheters and other intravascular devices are advanced along the same oblique approach angle. FIG. 4 is an illustration similar to FIG. 3 with the addition of all indwelling guidewire 16 as may remain after the endovascular procedures have been completed and the last of the endovascular catheters and the introducer sheath have been removed from the patient. At this point in the procedure, it is necessary to close the arteriotomy.

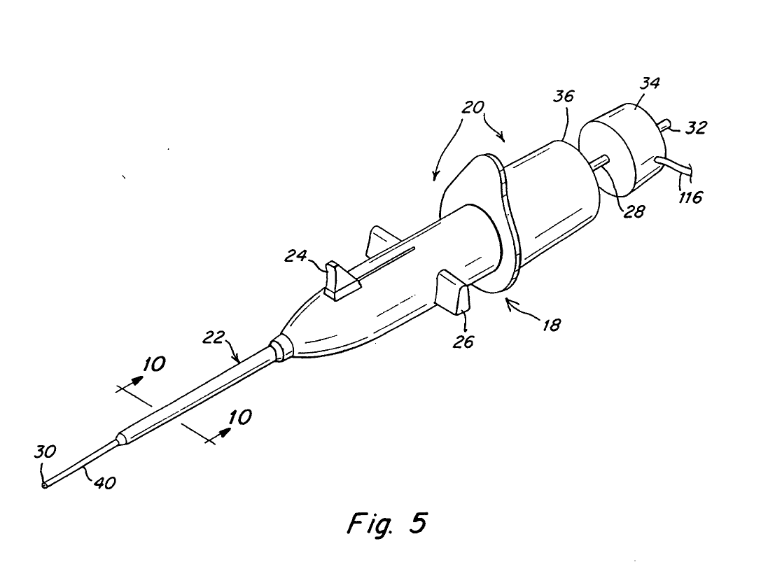

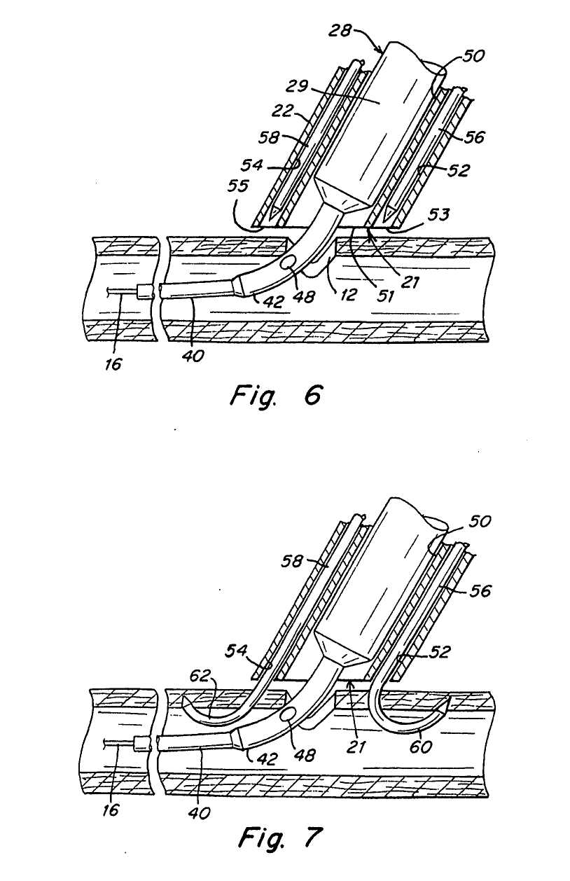

The present invention relates to a device and technique for placing a tubular guide sheath through the needle track, indicated in phantom at 17 in FIG. 4, to provide a guideway through which an arteriotomy closure device can be advanced and guided directly to the site of the arteriotomy 12. FIG. 5 illustrates generally, and in a simplified diagrammatic form, a tubular placement device 18 that may be used for that purpose and which may incorporate principles of the invention. The tubular placement device 18 includes a proximal control handle 20 and a guide sheath 22 that extends distally from the control handle 20. The distal end of the guide sheath may be considered as having a distal face 21 that preferably is formed it an oblique angle to the longitudinal axis of the sheath, as shown in FIG. 6. The handle may have various control elements 24, 26 by which the various functions of the device can be controlled and the orientation of the sheath can be determined.

The device 18 also includes a blood marking tube 28 that extends through the handle 20 and the guide sheath 22. The distal end 30 of the blood marking tube 28 protrudes distally beyond the distal end of the guide sheath 22 and the more proximal portions of (he blood marking tube 28 extend through the control handle 20 an(d may be attached securely to a proximal knob 34. The knob 34 is shown in FIG. 5 as being partly separated from the proximal end 36 of the handle 20. Initially, the blood marking tube 28 will be maintained in its most distal position in which the knob 34 is in abutment with the proximal end 36 of the handle. The blood marking tube 28 may be removed entirely from the device 18 by grasping the knob 34 and withdrawing the knob and tube from the control handle 20. As described in further detail below, when the knob 34 abuts the proximal end of the handle, tile relative position of the guide sheath 22 and the blood marking tube 28 is fixed.

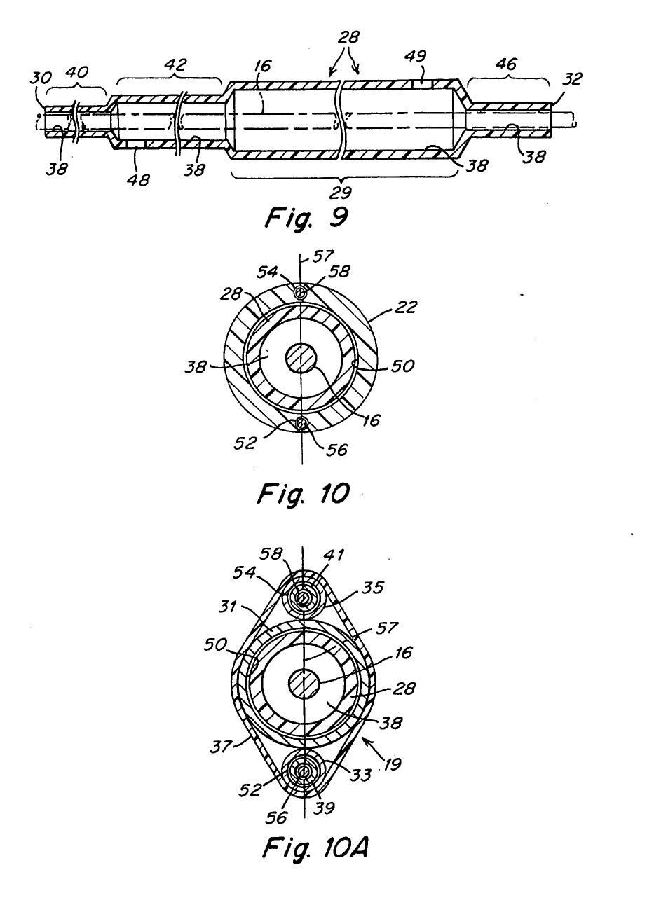

FIG. 9 illustrates the blood marking tube 28 in more detail. The tube may be formed from any of a variety of polymeric materials commonly used for catheters, dilators, intravascular sheaths and the like such as, for example, polyethylene, polyamide, polyolefin, fluoropolymer and polyethylene block amide copolymer, with lower durometer resins used for the more distal segments where increased flexibility may be desired. A lumen 38 extends the full length of the tube. The main segment 39 of the length of the blood marking tube 28 is dimensioned to fit slidably within a receptive central lumen in the guide sheath. The distal region of the blood marking tube 28 includes a smaller diameter distal tip segment 40 and an intermediate segment 42 with an outer diameter between those of the tip segment 40 and the main segment 39. The lumen 38 that extends through the tube 28 is adapted to receive a guidewire, such as the indwelling guidewire 16. The portion of the lumen 38 in tip segment 40 of the tube 28 preferably has it diameter just large enough to slidably accept the guidewire 16 with minimal fluid leakage. The proximal end of the blood marking tube 28 may have a proximal segment 46 of reduced diameter, also with the lumen 38 just large enough to slidably receive the guidewire 16 with minimal leakage. The blood marking tube includes a distal blood marking port 48 formed in tile intermediate segment 42 of the tube 28. The inner diameter of the portion of the blood marking tube lumen 38 that extends through the intermediate segment 42 leaves ample annular space for blood to flow into the blood marking port 48 and through the lumen 38, about the guidewire and through the main segment 29 of the tube 28. A proximal blood marking port 49 may be formed at the proximal end of the main segment of tube 28 to enable the clinician to observe the presence or absence of blood flow through the tube 28, thereby determining whether the distal port 48 is positioned in the blood vessel. The relative positions of the distal port 48 and the distal face 21 of the guide tube 22 are fixed such that the presence of blood flow confirms proper positioning of the distal face 21 of the guide sheath 22 relative to the arteriotomy.

FIG. 10 illustrates, diagrammatically, a simplified form of the internal construction of the guide sheath 22 and the blood marking tube 28. The guide sheath 22 may be formed from any of a variety of materials commonly employed for catheters and sheaths such is polyethylene, or PTFE, among others. In one arrangement the guide sheath may be extruded to form a central lumen 50 and a pair of wire lumens 52, 54. Each of the lumens 50, 52, 54 terminates in a distal outlet opening 51, 53, 55 respectively at the distal face 21 of the guide tube 22, as shown in FIG. 6. The wire lumens 52, 54 are located in diametrically opposite positions with respect to the central lumen 50. In the illustrated embodiment the central lumen 50 and wire lumens 52, 54 are aligned along a central plane 57 (FIGS. 10, 10A) so that the distal end of the guide sheath 22 can be located adjacent to the arteriotomy with the three lumens 50, 52, 54 extending along the midline plane 13 of the blood vessel and with the distal openings 53, 55 facing the vessel wall proximally and distally, respectively, of the arteriotomy 12. Preferably, the distal openings may be circumferentially spaced about 180 degrees. So positioned, the central plane 57 of the sheath is in general alignment or coincident or parallel with the midline plane 13 of the vessel. The distal end of the sheath may be formed to define a face 21 lying at an oblique angle corresponding to the approximate expected angle of approach. As shown in FIG. 8A, the oblique end face 21 may be oval and may be considered as having a major axis 57 and a minor axis 59. The central lumen 50 and tile wire lumens 52, 54 preferably are disposed along a plane that includes the major axis 57.

The wire lumens 52, 54 are adapted to slidably contain retention wires 56, 58, respectively. The distal tips of the retention wires are sufficiently sharp to be able to pierce the arterial wall. The wires 56, 58 may be formed from any of a number of biocompatible metals, for example, nitinol or spring steel in forms that enable the distal ends to assume their anchor configuration when they emerge from their respective lumens as well as to return to a linear configuration when withdrawn into and constrained within the lumens. The wires should(have sufficient column strength to be pushed through the lumens and may have supplemental support as described below.

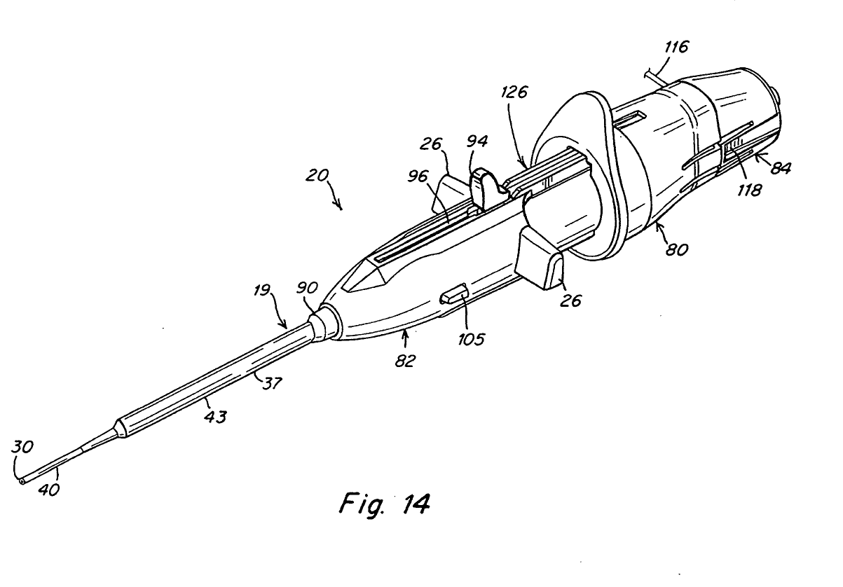

FIG. 10A illustrates a more detailed and preferred arrangement for the guide sheath. The lumens 50, 52, 54 are formed from several tubes arranged in a tube assembly 19 that includes a center tube 31 that defines the central lumen 50 and a pair of side tubes 33, 35 that are attached to the center tube 31 and define the wire lumens 52, 54. The center tube 31 and side tubes 33, 35 may be contained within and secured together by a polymeric transition sheath 37. The tube assembly 19 is covered by a thin, protective sheath 43 that protects the tube assembly 19 and distal ends of the tubes 33, 35, as shown in FIG. 14. The protective sheath 43 is retractable to expose the distal ends of the tubes 33, 35. The diamond-like cross-sectional shape of the transition sheath 37 shown in FIG. 10A is considered to enhance the ability of the device to track through tissue. As with the previously described embodiment of the sheath, the distal face of the sheath is formed at an oblique angle relative to the longitudinal axis of the sheath, preferably with the side tubes 33, 35 lying along the major dimension of the distal face of the sheath with the central lumen disposed between the side tubes.

FIG. 10A also illustrates all arrangement for reinforcing the wires 56, 58 to reduce the risk of those wires buckling when advanced in a distal direction. To that end, the wires 56, 58 are contained within support tubes 39, 41 that are secured to the wires, as by crimping, so that the support tubes may move with and reinforce the wires 56, 58 as they are advanced, together, within the side tubes 33, 35. The support tubes extend distally together with their respective wires but terminate short of the distal tips of the wires. The distal end of each wire protrudes distally beyond the distal end of its support tube 39, 41 by an amount sufficient to enable the wires to pierce the wall of the blood vessel and form their respective anchoring configurations. By way of dimensional example, for wires 56, 58 of about 0.016 inch diameter, the support tubes may have an inner diameter of about 0.0195 inch and an outer diameter of about 0.032 inch, the side tubes may have inner and outer diameters of about 0.038 and about 0.050 inch, respectively, and the center tube may have inner and outer diameters of about 0.140 and 0.156 inch, respectively, to receive a blood marking tube 28 having all outer diameter of the order of 0.130 inch. The center tube, support tubes and side tubes may be formed from stainless steel.

FIG. 6 illustrates, diagrammatically, the distal end of the guide sheath 22 and blood marking tube 28 when they have been advanced onto and along the indwelling guidewire 16 to place the distal end 21 of the sheath 22 adjacent and in registry with the arteriotomy 12. The distal blood marking port 48 will be spaced from the end of the sheath 22 by an amount slightly greater than a typical thickness of the arterial wall at the puncture site, for example of the order of two to four millimeters. Thus, when the assembly is advanced along the indwelling guidewire 16, entry of the blood marking port 48 into the lumen of the artery will be accompanied by blood flow through the port 48 and the lumen 38 of the blood marking tube 28 to enable the clinician to observe the flow of blood through the proximal blood marking port 49. That indicates to the clinician that the distal end 21 of the guide sheath 22 is adjacent and in registry with the arteriotomy. It should be noted that by locating the distal blood marking port 48 in the reduced diameter intermediate segment 42, the blood marking port 48 can enter the vessel lumen without the tube 28 causing further dilation of the arteriotomy. By way of illustrative example the intermediate segment 42 may be of the order of about 0.066″ outer diameter. The distal tip segment 40 may be about 0.042″ diameter; the main segment about 0.098″ diameter and the proximal segment 46 about 0.040″ diameter. The inner diameters of the lumen 38 have similarly varied dimensions so that the flow area between the blood marking ports 48, 49 will be unobstructed by the guide wire 16.

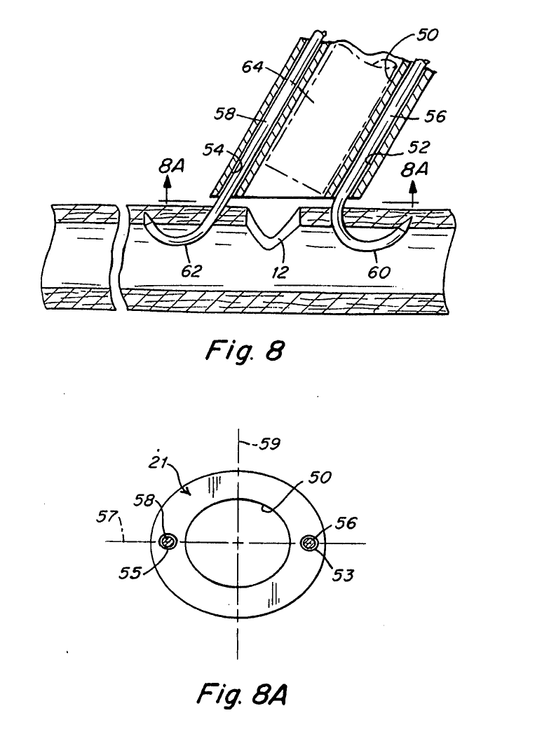

Once the distal end 21 of the guide sheath 22 is adjacent and in registry with the arteriotomy, the retention wires 56, 58 then are advanced distally and out of their respective wire lumens 52, 54 in order to stabilize the sheath to maintain its position relative to the arteriotomy as suggested in FIG. 8. The distal portions of the retention wires are formed with pre-set, non-linear anchors, such as curves 60, 62 that the retention wires will progressively assume as they emerge out of their respective lumens 52, 54. The distal ends of the wire are sharp and will pierce the wall of the vessel while forming into their curved anchor configurations. In the illustrative embodiment of FIG. 8 the pre-set curves are shown, diagrammatically, as J-shaped hooks although, it should be understood that other shapes for the anchors may be employed provided that they can be retracted into their respective wire lumens while reverting to a linear configuration as shown in FIG. 6. As shown in FIG. 7, the pre-formed shapes of the curves may be different to accommodate the different paths they should take in order to each securely engage the wall of the blood vessel. Thus, the more distal retention wire 58 may not require as sharp a curve 62 as the curve 60 on the more proximal retention wire 56. This results from the oblique approach angle described above. In the illustrative embodiment the curves 60, 62 preferably may be such as to enable the sharp tips to pierce the vessel wall, pass through the vessel wall and embed their tips in tile vessel wall as shown. Thus, as shown in FIG. 7, when the wires emerge, they will pierce the vessel wall while also assuming their pre-formed curves that form hooks adapted to engage the artery and stabilize the position of the sheath 22. The clinician may assess the proper engagement of the retention wires with the vessel by applying a slight tug on the device while observing the flow of blood from the proximal blood marking port. The continued presence of blood flow at the blood marking port during or after such testing confirms that the tubular placement device 18 is properly attached to the artery. The blood marking tube 28 and indwelling guidewire 16 then may be removed through the sheath 22 and an arteriotomy closure device, shown in phantom at 64 in FIG. 8, may be advanced through the central lumen 50 to the region of the arteriotomy 12 where its closing function may be performed. After or while the closure device performs its function to close the arteriotomy, the retention wires 56, 58 may be withdrawn proximally into their respective lumens 52, 54, the curved, flexible hook portions 60, 62 becoming straightened as they retract into their lumens.

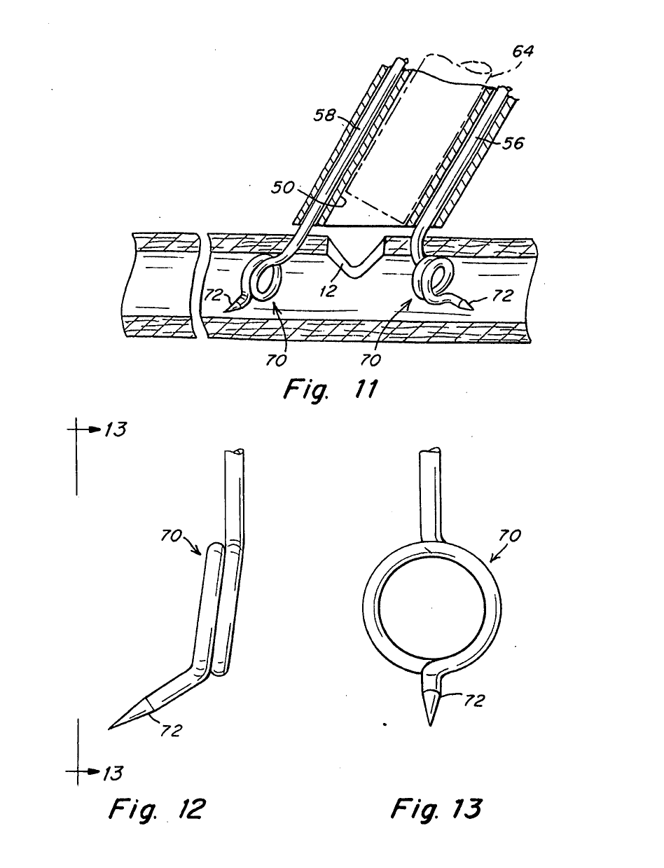

FIGS. 11-13 illustrates an arrangement similar to FIG. 8 but in which the anchors are provided with a different configuration. In this arrangement, the distal ends of the wires are formed to define a generally flat, somewhat planar coil 70 with the distal-most segment 72 of the wire extending in a direction orthogonal or at a substantial angle to the plane of the coil 70. When the device is advanced into position to deploy the wires, their sharp distal tips are extended to pierce the tissue and then, as they advance into the lumen of the blood vessel, the distal ends revert to their coiled configuration. The sharp distal tip does not re-enter the wall of the blood vessel and is maintained within the mid-region of the vessel lumen to avoid injury to the vessel wall. The planar coils should be configured so that when they have deployed and formed within the vessel, the distal tips of the wires are maintained in the mid-region of the vessel with their curved segments of the loops 70 in engagement with the vessel wall on opposite sides of the arteriotomy. It should be understood that other anchoring configurations may be employed in the practice of the invention.

The operational sequence of the elements of the tubular placement device 18 may be controlled at the handle 20 on the proximal end of the device. The device initially is in the configuration in which the distal tip segment 40 of the blood marking tube 28 protrudes distally beyond the distal end of sheath 22 illustrated in FIG. 5, or beyond the distal end of the tube assembly 19 and the retractable protective sheath 43 illustrated in FIG. 14. After the clinician has withdrawn the procedural catheters and introducers (not shown) from the patient, leaving the guidewire 16 in place, the device 18 then is back-loaded over the proximal end of the guidewire by inserting the proximal end of the guidewire into the opening of the guidewire lumen at distal end 30 of the blood marking tube 28. The device is advanced until the distal blood marking port has entered the vessel as confirmed by the presence of blood flow from the proximal blood marking port. The rotational orientation of the sheath with respect to the blood vessel and the puncture wound may be adjusted by reference to visual elements at the proximal end of the device, for example, as by reference to the orientation of the handle itself and distinct components of the handle 20, described below. This provides assurance that the distal end of the tube assembly 19 or sheath 22 is located in proximity to the outer surface of the vessel in the region of the arteriotomy. In the preferred embodiment, the outer protective sheath 43 then is withdrawn proximally over tube assembly 19 to expose the distal ends of the retention wire lumens 52, 54. The retention wires 56, 58 then are advanced distally to cause each wire to pierce and pass through the vessel wall, one wire being distal of the arteriotomy and the other proximal. In the ideal situation where the arteriotomy is centered laterally in the artery, as shown in FIG. 1, both retention wires pierce the vessel wall close to or at the medial plane of the vessel. As the distal ends of the wires pass through the vessel wall, they will assume their anchor configurations to engage the arterial wall proximally and distally of the arteriotomy. The clinician may assess the proper engagement of tile retention wires with the vessel by applying a slight tug on the device while observing the flow of blood from the proximal blood marking port. The continued presence of blood flow at the blood marking port during or after such testing confirms that the tubular placement device 18 is properly attached to the artery. Should the blood flow diminish or stop, that is an indication that the blood marking port has been withdrawn out of the blood vessel. In order to reposition the device, the retention wires are retracted into their respective lumens, the device is advanced over the guidewire to reposition the distal blood marking port within the vessel and the retention wires are again advanced distally to pierce the vessel wall and engage the anchors. When it hats been determined that the device is positioned and anchored properly, the blood marking tube and guidewire may be removed from the device, clearing a passageway through the central tube through which an arteriotomy closure device may be advanced to close the arteriotomy. The arteriotomy closure device may take any of a number of forms adapted to close all arteriotomy. After the arteriotomy has been closed, the retention wires are retracted with the anchors reassuming their linear configuration within their respective lumens and the entire device then may be removed from the patient.

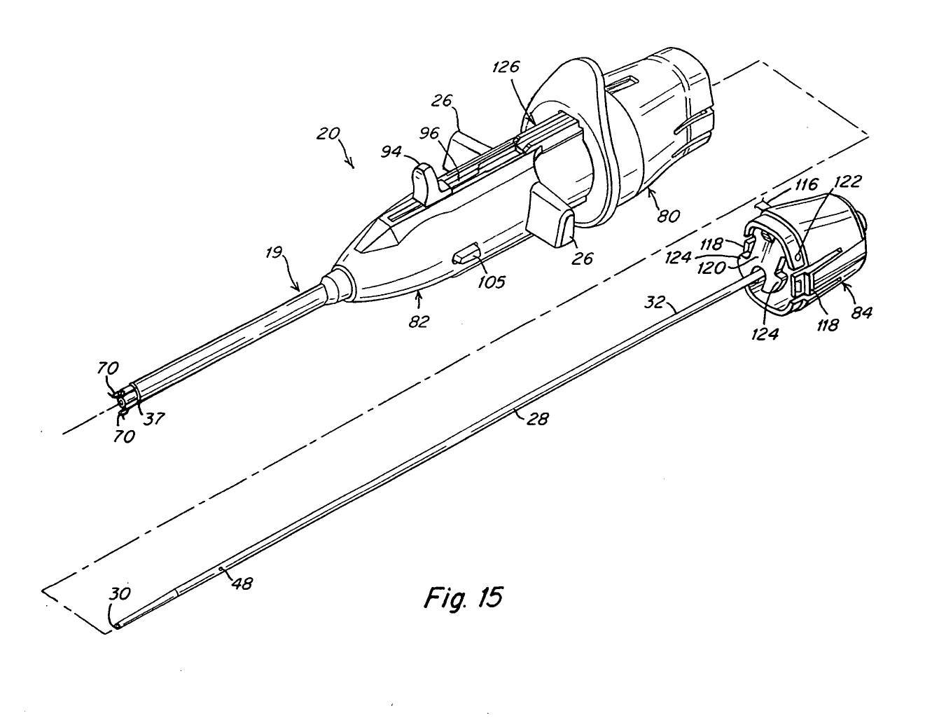

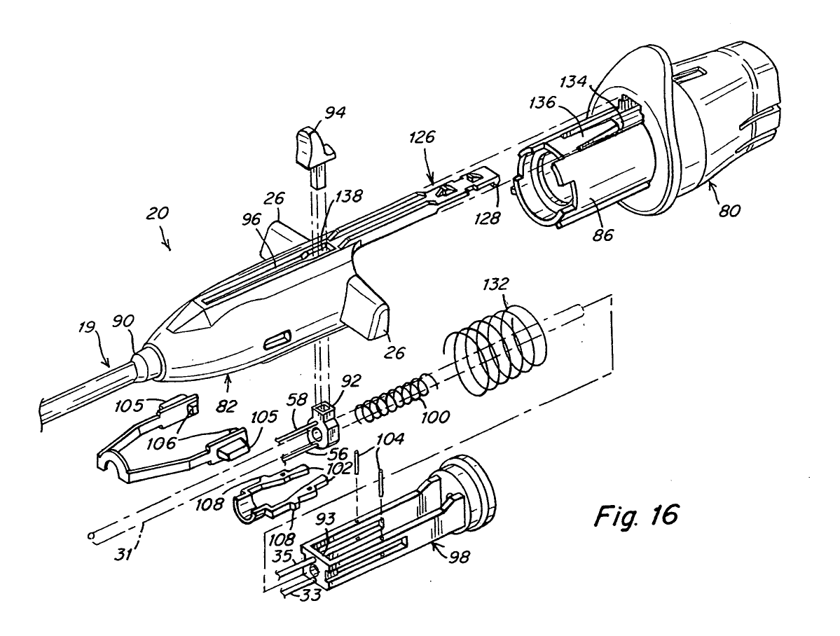

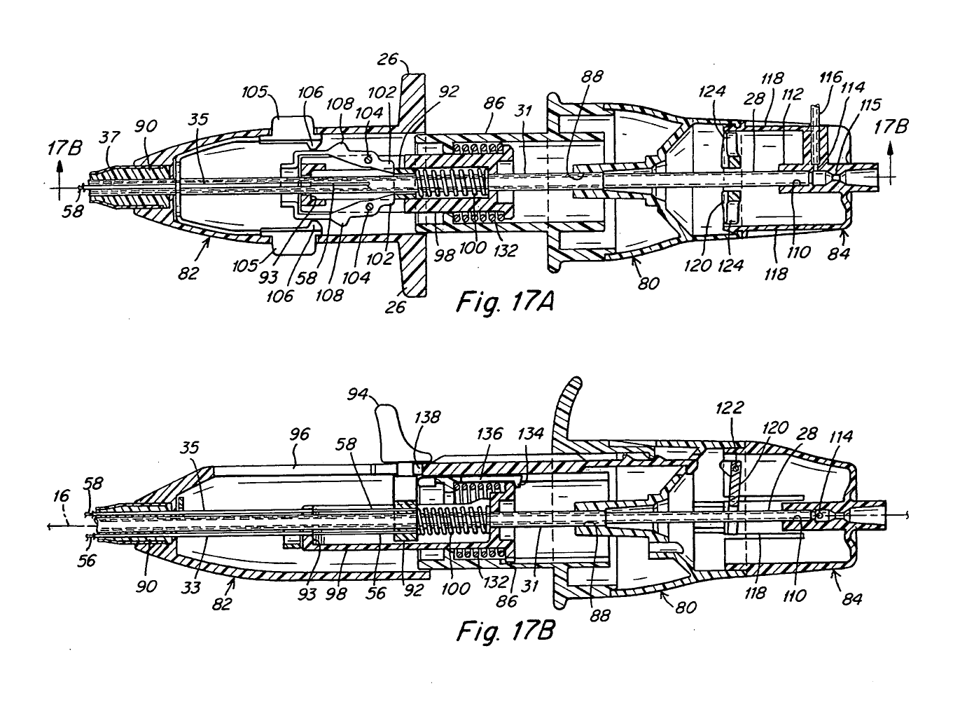

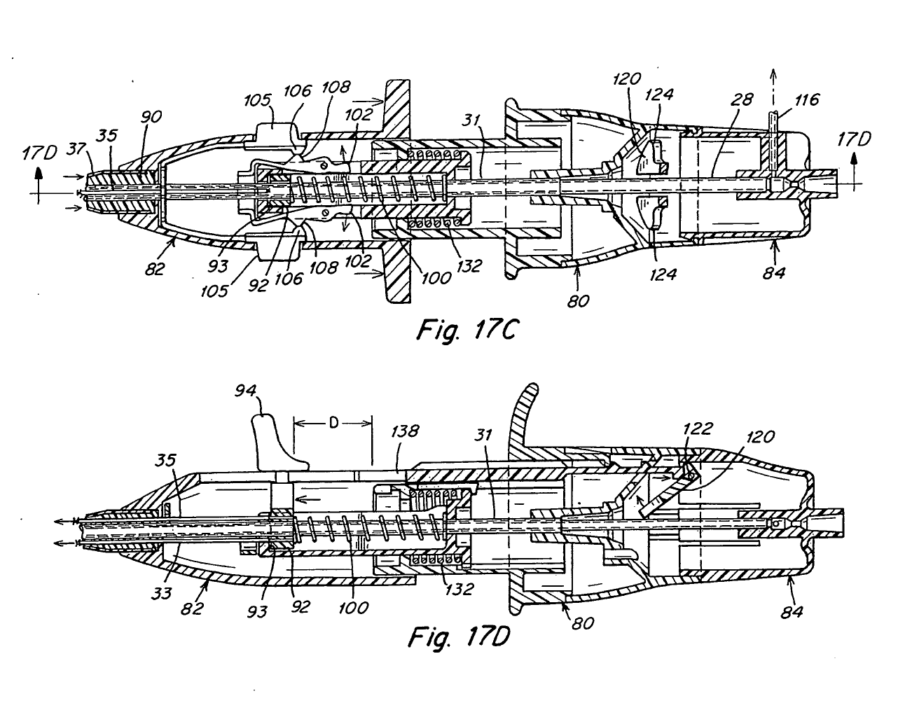

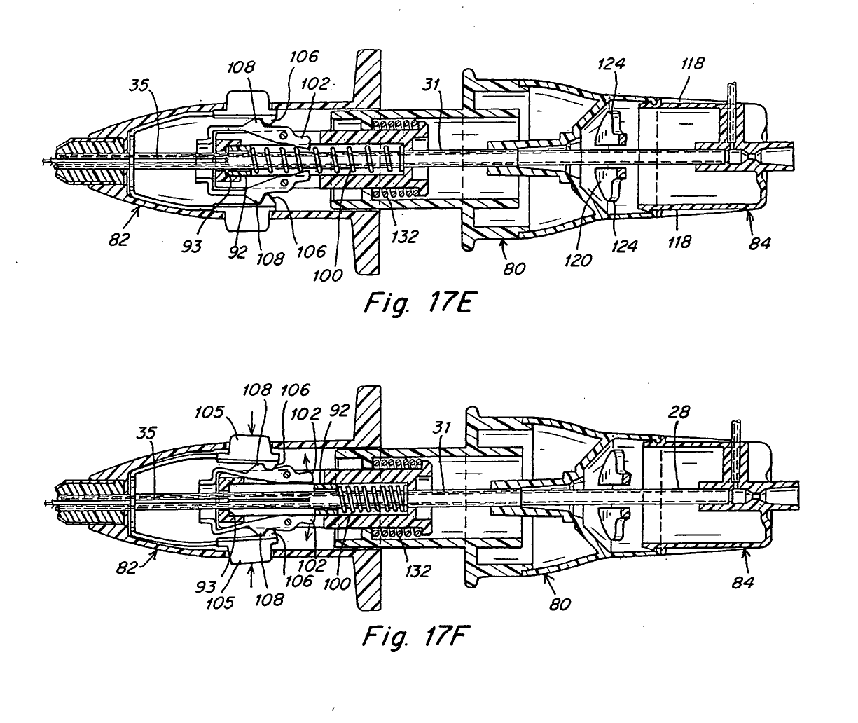

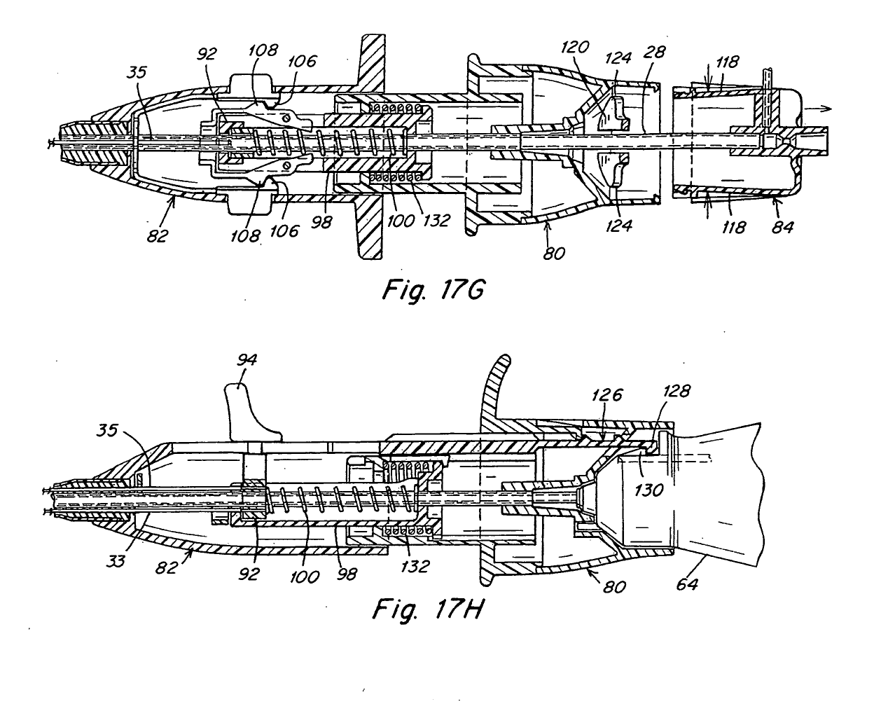

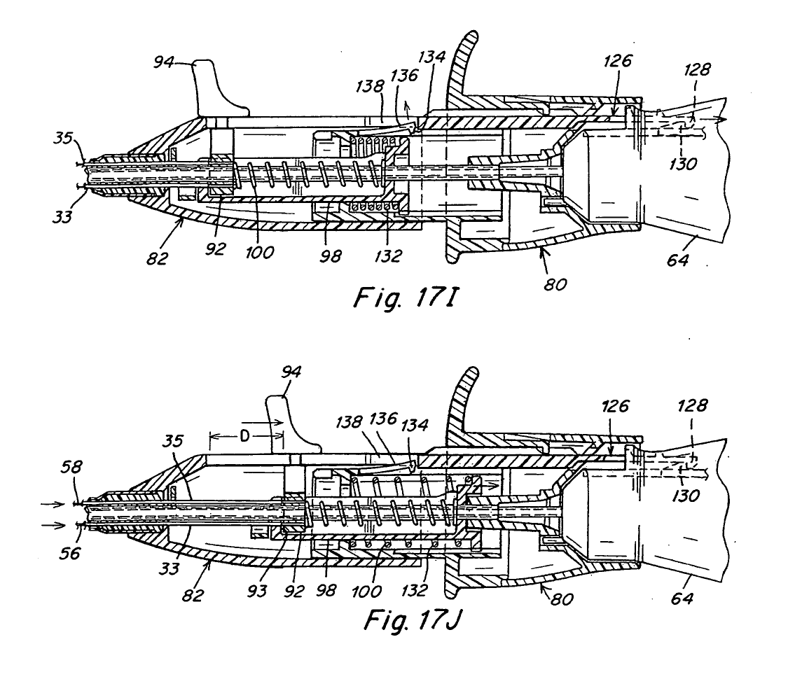

FIGS. 14-16 illustrate a preferred embodiment of the handle 20 by which the device may be controlled in a sequence illustrated in FIGS. 17A-17J. The handle 20 may be considered as having three principal sections including a base 80, a retractor assembly 82 and a proximal cap 84, corresponding to knob 34 in FIG. 5. The retractor assembly 82 is hollow and is slidable longitudinally on a distally extending portion 86 of the base, the retractor 82 having laterally projecting control tabs 26 to facilitate retraction. The tubing assembly 19 is mounted securely to the base 80, as by press-fitting the proximal end of the center tube 31 into a socket 88 formed as part of the base 80 (FIG. 17A). The side tubes 33, 35 extend proximally from the distal end of the tube assembly 19 to and into a fitting 90 at the distal end of the retractor assembly 82, the side tubes 33, 35 being secured to the center tube 31 as shown in FIG. 10A. The outer protective sheath 43 is attached to the fitting 90 at the distal end of the retractor assembly 82 and extends distally beyond the distal end of the tube assembly 19 to cover the distal ends of the side tubes. The proximal ends of the side tubes 33, 35 may extend through the fitting 90 to terminate within retractor assembly 82 at the distal end of carriage 98, to which they are secured.

The retention wires 56, 58 are slidable longitudinally through their receptive side tubes 33, 35 and are driven by a movable wire block 92 to which the proximal ends of each of the wires 56, 58 is secured. The central tube 31 extends through a receptive aperture in the wire block 92. The wire block 92 includes a manually accessible tab 94 that extends upwardly through a longitudinal slot 96 formed in the housing of the retractor assembly 82. The wire block 92 is slidably seated within an elongate carriage 98 housed within the hollow interior of the base 80 and the retractor assembly 82. When the device is in its ready-to-use configuration (FIGS. 17A, 17B), the wire block 92 and tab 94 are maintained in their most proximal position on the carriage 98 by a latch mechanism with the block 92 being biased distally by a compression spring 100. With the wire block 92 in its most proximal position, the retention wires 56, 58 are retracted into their respective tubes 33, 35 in readiness to be advanced to enable their distal ends to be projected out of their tubes and assume their anchor configurations. The wire block 92 is retained in its proximal, retracted position by a pair of pivot latches 102 mounted to the carriage 98 at pivots 104.

When a triggering member deploys the anchors, latches 102 are actuated to pivot about the pivots 104, thus releasing the wire block 92 to enable the compression spring 100 to drive the wire block, and the retention wires secured to the wire block, in a distal direction until the wire block 92 engages a stop 93 at the distal end of the carriage 98. To that end, the housing of the retractor assembly 82 may carry a pair of cams 106 arranged to engage followers 108 on the pivot latches 102. To trigger the device, the retractor assembly 82 is withdrawn proximally over the distally extending portion 86 of the base 80 such that the cams 106 will engage the followers 108 to cause the latches 102 to pivot and release the wire block 92 for distal movement and deployment of the anchors (FIGS. 17C, 17D). The pivot latches 102 may be formed as a single molded plastic member as shown. Similarly, the cams 106 also may be molded as a single plastic element. The cams 106 may be flexed radially inwardly as described below.

Should the clinician determine, after triggering the device, that the anchors have not stabilized the position of the device, the wires 56, 58 may be retracted into their respective side tubes 33, 35 by sliding the wire block tab 94 proximally in longitudinal slot 96. That draws the wire block 92 proximally through the pivot latches 102 which spread apart to permit proximal movement of the wire block and then snap into place into engagement with a forward facing surface of the wire block (FIGS. 17E, 17F). With the retention wires retracted, the clinician may reposition the device and then reactuate the pivot latches 102. Because the housing of the retractor assembly 82 will have been withdrawn proximally over the distally extending portion 86 of the base 80, the cams 106 are located in a more proximal position than when the device was triggered but remain in engagement with the pivot latches 102 distally of the pivots. In this configuration, the pivot latches 102 may be reactuated (FIGS. 17G, 17H) to advance the retention wires 56, 58 by pressing in the cams 106 that have portions 105 protruding radially outwardly through side apertures formed in the retractor assembly housing. The retraction and re-advancement of the retention wires by sliding tab 94 and pressing cam portions 105 can be repeated until the clinician is satisfied that the tubular placement device 18 has been anchored to the vessel. The sliding tab 94, cams 106 and the associated components described above comprise a control member that provides for the retraction and extension of the retention wires between their retracted and deployed positions.

The blood marking tube and guidewire may be removed to provide passage through the central tube 31 for the arteriotomy closure device. In the control handle illustrated, the proximal end of the blood marking tube 28 is mounted in a receptive socket 110 formed in a nipple 112 that may be formed integrally with the proximal cap 84. In the embodiment illustrated in FIG. 17A, the proximal end of blood marking tube 28 lacks reduced diameter proximal segment 46 and proximal blood marking port 49. Socket 110 includes a stop for abutting the proximal end of marking tube 28 and a reduced diameter orifice 115 that opens to the exterior of the cap 84. The orifice 115 is functionally similar to the internal lumen 38 through proximal segment 46 of the blood marking tube 28. That is, the orifice 115 is just large enough to slidably receive the guidewire 16 with minimal leakage. A side port 114 is formed in the socket 110 to between the stop and the orifice 115. A short outlet tube 116 may be connected to the port 114 and extend out of the cap 84. The open proximal end 32 of blood marking tube 28 is functionally similar to proximal blood marking port 49. When the distal blood marking port 48 is positioned within the vessel, blood enters lumen 38 and exits the proximal end 32 of blood marking tube 28 through socket 110, port 114 and outlet tube 116 to enable the clinician to observe blood flow. In an alternative embodiment (FIG. 5), proximal portion 32 of blood marking tube 28 extends through cap 84 such that reduced diameter proximal segment 46 protrudes from cap 84. In this embodiment, the short outlet tube 116 may be fluidly connected to proximal blood marking port 49 to enable the clinician to observe blood flow.

The blood marking tube 28 is withdrawn by grasping the proximal cap 84 and retracting it to draw the blood marking tube together with the guidewire out of the patient and the sheath 22 or center tube 31. As mentioned above, the relative positions of the blood marking tube 28 and the guide sheath 22 are fixed such that the presence of blood flow confirms proper positioning of the guide sheath 22 relative to the arteriotomy. In order to avoid disturbing this relationship by inadvertent detachment of the proximal cap 84 and blood marking tube, a latching mechanism is provided to assure that the cap cannot be removed from base 80 until after the anchors are deployed and in proper engagement with the vessel. To that end, the proximal cap 84 is formed to include a pair of flexible integral fingers 118 on opposite sides of the cap, the free distal ends of the fingers 118 being formed to have snap-fit latching elements engageable with corresponding mating latching elements formed on the proximal end of the base 80.

The proximal cap 84 is detached simply by squeezing the fingers 118 and withdrawing the cap. As an additional safety mechanism to prevent inadvertent cap detachment, a safety locking member 120 is pivoted internally of and to the cap about pivot 122. The safety lock 120 has laterally projecting fingers 124 that, when in engagement with the inner surface of the fingers 118, prevent the fingers from being squeezed inwardly. The safety lock 120 is actuated to pivot the fingers 124 forwardly and out of engagement with the locking tabs 118 by a rearwardly extending, elongate arm 126 that may be formed as part of the retractor assembly 82 (FIGS. 17C, 17D). The arm 126 is configured so that its proximal end will trip the safety lock 120 to pivot the arms 126 out of engagement with the fingers after the wires have pierced the vessel and the anchors have formed.

The tubular placement device 18 also includes an arrangement to maintain it in position until the arteriotomy closure device has performed its function. To that end, the device may be arranged to require cooperation with the arteriotomy closure device in order to cause retraction of the retention wires into their respective side tubes 33, 35. That may be achieved by mounting the carriage 98 in the base 80 to have additional rearward motion such that the carriage can draw the wire block 92 rearwardly in response to cooperation with the arteriotomy closure device. In the illustrative embodiment, the proximal end of the arm 126 is provided with a shoulder 128 that is engageable with a cooperative shoulder 130 (FIGS. 17H-17J) associated with the mechanism 64 for delivering the arteriotomy closure. When the arteriotomy closure device is advanced through the central tube, the shoulder 130 on the closure device interlocks with the shoulder 128 on the guide device so that when the deployment mechanism on the closure device is actuated (e.g., by a trigger) the interlocked shoulders 128, 130 are drawn rearwardly to draw the arm 126 and the retractor assembly 82 toward its most rearward position. Rearward movement of the arm 126 causes release of the carriage 98 to permit further rearward movement under the influence of a compression spring 132 arranged to bias the carriage rearwardly when the carriage is permitted such movement. The carriage 98 is held in its intermediate position by a restraining tab 134 formed at the end of a flexible finger 136. The tab 134 engages the rearward end of the carriage to prevent it from moving rearwardly under the influence of the compression spring 132.

The rearwardly extending arm 126. is provided with a window 138 that will be positioned over the finger 136 and restraining tab 134 as the arm 126 is drawn rearwardly by the arteriotomy closure device. When the window 138 is aligned with the distal portion of the arm, the arm, which may be molded to the base 80 to be biased upwardly, can spring into the window 138 sufficiently to release the proximal end of the carriage 98. That enables the spring 132 to drive the carriage rearwardly, carrying with it the wire block 92 and retention wires. With the retention wires drawn into their respective side tubes 33, 35, the device is no longer anchored to the vessel and may be removed.

It should be understood that the foregoing description of the invention is intended merely to be illustrative thereof and that other embodiments, modifications and equivalents may be apparent to those skilled in the art while remaining within the scope of the invention.

1. An apparatus for guiding a puncture closure device to a percutaneous puncture in a blood vessel wall comprising:
an elongate sheath having proximal and distal ends and having a longitudinally extending central lumen receptive to a puncture closure device and a pair of longitudinally extending wire lumens, each of the lumens terminating in a separate distal port at the distal end of the sheath and the distal ports of the wire lumens being circumferentially spaced at opposing locations about a distal port of the central lumen, the ports each intersecting an imaginary plane extending centrally through the sheath;
each of the wire lumens containing a slidably movable retention wire, the distal end of each retention wire being sharp and adapted to pierce the wall of the vessel, the retention wires being slidable between a retracted position in which the distal ends are contained within their respective lumens and a deployed position in which the sharp ends are extended beyond the distal ports of their respective lumens;
a distal portion of each retention wire being preformed to a non-linear anchoring configuration when unconstrained, the anchoring configuration being adapted to resist detachment of the retention wire from the vessel wall when engaged with the vessel wall, the wire having sufficient flexibility to enable it to assume a linear configuration within its respective lumen when retracted into its lumen to its retracted position;
whereby the sheath may be located with its distal end adjacent and in registry with the puncture in the vessel and with the central plane of the sheath intersecting and extending longitudinally of the vessel, such that the retention wires can be advanced from their retracted positions to cause their sharp ends to engage and pierce the vessel wall at spaced locations proximally and distally of the puncture and to assume their anchoring configuration when engaged with the vessel wall to stabilize the sheath and central lumen in that position.

2. An apparatus as defined in claim 1 wherein the distal ports of the wire lumens are circumferentially spaced about 180 degrees.

3. An apparatus as defined in claim 1 further comprising a region of each retention wire, proximal of its anchor portion, being contained within a reinforcement tube to enhance the column strength of the wire.

4. An apparatus as defined in claim 1 wherein the central lumen is defined by a center tube and the wire lumens are defined by smaller diameter tubes attached to the central tube; and
a transition sheath disposed about the tubes to provide a smooth external surface about the central and wire tubes.

5. An apparatus as defined in claim 4 wherein the smaller tubes and center tube extend along a common plane and the transition sheath is approximately diamond shaped in cross-section.

6. An apparatus as defined in claim 1 further comprising visual means at its proximal end by which the orientation of the sheath and the distal ports of the lumen may be determined.

7. An apparatus as defined in claim 1 wherein the elongate sheath has a distal face disposed at an oblique angle to the length of the sheath, the distal face having major and minor dimensions, the distal ports of the central and wire lumens being located at the distal face, the distal openings of the wire lumens and the central lumens being aligned along the major dimension of the distal face.

8. An apparatus as defined in claim 1 further comprising:
a handle mounted to the proximal end of the sheath, the handle having a visual indicator oriented to facilitate orientation of the distal end of the sheath with the vascular puncture.

9. The apparatus as defined in claim 8 wherein the handle includes a control member operatively associated with a proximal portion of each of the retention wires whereby the control member may be operated to extend or retract the retention wires between their retracted and deployed positions.

10. The apparatus as defined in claim 1 further comprising:
a blood marking tube slidable within the central lumen of the sheath and having a blood marking lumen extending along its length, the blood marking tube having a proximal and a distal blood marking port adjacent each of the respective proximal and distal ends of the blood marking tube, the blood marking ports being in communication with the blood marking lumen;
tile blood marking tube being slidable with respect to the sheath between a distal-most position in which the distal blood marking port is disposed a fixed distance from the distal end of the sheath and a position in which the blood marking tube is removed entirely from the central lumen.

11. The apparatus as defined in claim 10 further comprising:
a control handle mounted to the proximal end of the sheath;
the blood marking tube comprising a tube having a length greater than the combined length of the handle and the sheath, the blood marking tube having a member attached thereto and adapted to abut a portion of the handle to limit the extent of distal protrusion or the tube with respect to the sheath;
the blood marking tube having a distal tip segment, a larger diameter intermediate segment proximal of the distal segment, a main segment larger in diameter than the intermediate segment and disposed proximal thereto and a proximal segment smaller in diameter than the main segment, the portion of the blood marking lumen that extends through the proximal and distal segments being closely dimensioned to a guidewire adapted for use with the device to permit sliding of the guidewire through the lumen but to resist blood flow through the proximal and distal segments, the distal segment and at least a portion or the intermediate segment extending distally beyond a distal face of the sheath when the blood marking tube is in its most distal position, the blood marking lumen in the intermediate and main segments being substantially larger in diameter than the outer diameter of the guidewire; and
a distal blood marking port in that portion of the blood marking tube that extends distally beyond the distal face of the sheath and a proximal blood marking port located at the proximal end of the main portion of the blood marking tube, the blood marking port being fluidly communicated exteriorly of the handle to enable a clinician to observe blood emerging from the proximal port.

12. The apparatus as defined in claim 9 wherein the control handle contains a slidable wire block secured to the proximal ends of both of the retention wires;
spring powered means for biasing the wire block in a distal direction and a triggering member for releasing the block from a retracted position whereby the spring may drive the block and the retention wires in a distal direction.

13. The apparatus as defined in claim 12 wherein the handle further comprises a control operatively associated with the wire member for manually retracting the wire block to the retracted position in readiness to be released and advanced distally.

14. The apparatus as defined in claim 12 further comprising, means responsive to advancement of a puncture closure device through the lumen of the central tube for causing the wire block to be retracted and the retention wires attached thereto to be retracted proximally into their respective wire lumens.

15. A method for stabilizing a sheath for guiding a closure device to a percutaneous puncture in a blood vessel wall comprising:
providing a sheath having proximal and distal ends with a central lumen having a distal opening and adapted to receive a puncture closure device through the central lumen, the sheath having a pair of retention wires movable with respect to the sheath, each of the retention wires having a sharp distal end adapted to pierce the vessel wall and an anchor portion adjacent the distal end, the anchor portion being preformed with a non-linear configuration but having sufficient flexibility to enable the anchor portion to be constrained in a linear configuration when disposed within the sheath;
locating the distal end of the sheath to orient the distal opening of the central lumen in registry with the puncture of the vessel;
extending the retention wires to engage and pierce the vessel wall at spaced locations proximally and distally, with respect to the vessel puncture, the anchor portions assuming their non-linear configuration after the distal tips of the wires have pierced the vessel wall;
whereby the engagement of the anchor portions with the vessel wall stabilizes the position of the sheath with respect to the vessel puncture to enable the closure device to be guided by the sheath to the region of the vessel puncture.

16. A method as defined in claim 15 further comprising:
after the position of the sheath has been stabilized, advancing a puncture closure device through the central lumen of the sheath and operating the closure device to close the puncture.

17. A method as defined in claim 18 further comprising:
substantially simultaneously with the operation of the puncture closure device, retracting the retention wires into their linear configurations within the sheath; and
removing the sheath and the closure device from the patient.
